(19) United States
(12) Patent Application Publication
(10) Pub. No.: US 2024/0060025 A1
(43) Pub. Date: Feb. 22, 2024

(54) LARGE CELLULAR MICROCOMPARTMENTS COMPRISING A PLURALITY OF CYSTS

(71) Applicant: TREEFROG THERAPEUTICS, PESSAC (FR)

(72) Inventors: Maxime FEYEUX, TALENCE (FR); Andrea LEONARD, BORDEAUX (FR); Philippe COHEN, BLANQUEFORT (FR)

(21) Appl. No.: 18/270,931

(22) PCT Filed: Jun. 16, 2022
(86) PCT No.: PCT/EP2022/066498

§ 371 Date: Jul. 5, 2023

(30) Foreign Application Priority Data
Jun. 16, 2021 (FR) FR2106403
Dec. 31, 2021 (FR) FR2114709

(51) Int. Cl. C12M 1/00; C12M 1/12; C12M 3/06; C12N 5/074

(52) U.S. Cl. CPC C12M 23/34; C12M 23/16; C12M 25/14; C12N 5/0696

(57) ABSTRACT

The invention relates to a three-dimensional cellular microcompartment or an assembly of three-dimensional cellular microcompartments of ovoid, cylindrical, spheroid or spherical shape, or substantially ovoid, cylindrical, spheroid or spherical shape, comprising an external hydrogel layer defining an internal part (14), said internal part comprising at least:
extracellular matrix elements, and
at least two cysts, each cyst being formed by at least one layer of human or animal cells organised three-dimensionally around a lumen,
the smallest radius or the average radius of the internal part being at least 100 μm. The invention also relates to a method for producing such a microcompartment or microcompartment assembly.

### TECHNICAL FIELD

The invention relates to the three-dimensional culture of epithelial-type cells, such as pluripotent stem cells.

### PRIOR ART

Ex vivo cell culture is a field which is generating increasing interest. The cultured cells may be of any type. It may involve differentiated cells with different phenotypes, progenitor cells and stem cells. A significant advance in cell culture techniques is the introduction of three-dimensional culture systems.

Indeed, three-dimensional cultures are advantageously closer to natural in vivo systems, and can be used for numerous applications, in particular in the development of therapies. A particularly suitable technology is that described in application WO2018/096277 which consists of three-dimensional cellular microcompartments for culturing stem cells.

However, despite their efficiency, existing 3D culture systems still have limitations in terms of yield and growth rate of the cells to get even closer to in vivo expansion rates and cycle lengths while ensuring a stable epithelial phenotype is maintained.

The objective of the invention is to propose a three-dimensional cell culture solution satisfying all of these needs and overcoming the disadvantages and limits of the prior art for an even more quantitative, and still at least equally qualitative, culture.

### SUMMARY OF THE INVENTION

While working on the development of cellular microcompartments for the 3D culture of epithelial cells or cells having epithelial-type morphology and which are able to form cysts, such as pluripotent stem cells, the inventors have developed a system making it possible to increase the maximum number of cells contained in a microcompartment organised around a lumen (cyst), while maintaining an epithelial phenotype

According to the invention, the maintenance of a weak seeding of cells makes it possible to increase the amplification factor between the seeding of the cells in the microcompartment and the harvesting of the microcompartment containing the amplified cells. In existing systems, microcompartments comprising a few cells at the time of seeding (1 to 3 in particular) die or restart their growth with a latency rate which is detrimental to the yield of the culture and increases the necessary encapsulated culture time.

According to the invention, these problems are linked in particular to microcompartments whose size is too small, and in particular to microcompartments in which the volume of the internal part is too small.

Thus, the invention relates to a three-dimensional cellular microcompartment with an external layer and an internal part, the internal part of which has sufficiently large dimensions to allow a high growth rate and a large quantity of cells at the time of harvesting the microcompartment, starting from a low initial seeding of cells.

In particular, the invention targets a three-dimensional microcompartment of ovoid, cylindrical, spheroid or spherical shape, or substantially ovoid, cylindrical, spheroid or spherical shape, comprising an external hydrogel layer defining an internal part, said internal part comprising at least:
extracellular matrix elements, and
at least two cysts, each cyst being formed by at least one layer of cells organised three-dimensionally around a lumen,
the smallest radius of the internal part of the microcompartment being at least 100 μm, preferentially at least 200 μm.

The cells of each layer of cells organised three-dimensionally around a lumen are cells capable of forming a cyst, i.e. polarised cells with a basal surface capable of forming tight junctions and expressing podocalyxin on the apical surface (facing the lumen of the cyst). These are in particular epithelial cells or cells having a human or animal epithelial-type morphology. The cells of each layer of cells organised three-dimensionally around a lumen are preferentially chosen from induced pluripotent stem cells (iPSC) and the following cells: glandular epithelial cells (e.g. mammary or salivary), renal epithelial cells, intestinal epithelial cells (enterocytes), skin epithelial cells (keratinocytes), retinal pigment epithelial cells, epicardial cells, and endocardial cells.

Advantageously, such an arrangement, in particular the presence of at least two cysts, makes it possible to increase the maximum number of cells contained in a microcompartment while retaining an epithelial phenotype around a lumen (cyst). The size of the microcompartment according to the invention is chosen to allow the growth of several cysts while preserving a diffusion distance compatible with the physiology of the cells.

The invention also relates to a three-dimensional assembly of cellular microcompartments comprising at least one cellular microcompartment according to the invention, preferentially in liquid suspension in a bioreactor.

The microcompartments according to the invention may be useful for various applications and in particular in the prevention and/or treatment of pathologies.

The cellular microcompartments according to the invention can be obtained in particular by the implementation of a specific preparation method comprising the following steps:
(a) incubating cells in a culture medium, preferentially in a culture medium containing at least one cytoprotective factor, in particular an inhibitor of apoptosis and/or Rho/A kinases,
(b) mixing the cells from step (a) with extracellular matrix elements, in particular a biological or synthetic extracellular matrix,
(c) encapsulating the suspension of cells in a hydrogel layer so as to form a microcompartment of ovoid, cylindrical, spheroid or spherical shape or substantially ovoid, cylindrical, spheroid or spherical shape, comprising an external hydrogel layer defining an internal part, the smallest radius of said internal part being at least 100 μm;
(d) culturing the resulting microcompartments in an isotonic rinsing buffer, preferentially for less than 30 minutes, then in a culture medium, preferentially in a culture medium containing at least one cytoprotective factor, in particular an inhibitor of apoptosis and/or Rho/A kinases;
(e) preferentially rinsing the microcompartments, so as to remove the cytoprotective factor (inhibitor of apoptosis and/or Rho/A kinases), preferentially within 48 hours after encapsulation, even more preferentially within 24 hours;
(f) culturing the microcompartments for at least two cell division cycles (amplification), preferentially between 1 and 60 days, between 1 and 30 days, between 1 and 20 days, even more preferentially between 2 and 30 days, between 2 and 20 days, between 3 and 30 days, between 3 and 20 days, in particular between 4 and 7 days, in particular between 5 and 7 days, in a culture medium without a cytoprotective factor, and
(g) optionally recovering the resulting cellular microcompartments.

The method according to the invention makes it possible to obtain microcompartments according to the invention with at least two cysts.

Other features and advantages will emerge from the detailed description of the invention and the following examples.

### BRIEF DESCRIPTION OF THE FIGURES

FIG. 1a is a diagram of a microcompartment according to the invention comprising several cysts of induced pluripotent stem cells. This diagram is a representation of the microcompartment shown in the photograph of FIG. 1b.

FIG. 1b is a phase-contrast microscopy image of a microcompartment according to the invention.

FIG. 2a is a diagram of a series of microcompartments according to the invention.

FIG. 2b is a phase-contrast microscopy image of a series of microcompartments according to the invention.

FIG. 3a is a diagram of a bioreactor containing a series of microcompartments according to the invention.

FIG. 3b is an image of a bioreactor containing a series of microcompartments according to the invention.

FIG. 4a is a diagram of the fusion of two cysts in a microcompartment according to the invention.

FIG. 4b is an image of the fusion of two cysts in a microcompartment according to the invention.

FIG. 5 is a representation of the results of tests on the amplification of induced pluripotent stem cells in microcompartments according to the invention.

### DETAILED DESCRIPTION OF THE INVENTION

### Definitions

For the purposes of the invention, “alginate” means linear polysaccharides formed from β-D-mannuronate and α-L-guluronate, salts and derivatives thereof.

For the purposes of the invention, “hydrogel capsule” or “hydrogel microcompartment” means a three-dimensional structure formed from a matrix of polymer chains, swollen using a liquid, preferentially water.

For the purposes of the invention, “differentiated” cells means cells which have a particular phenotype, as opposed to pluripotent stem cells which are not differentiated, or progenitor cells which are undergoing differentiation.

For the purposes of the invention, “epithelial cells” or “epithelial-type cells” means human or animal cells associated with each other by intercellular junctions structuring a single epithelium (cuboid, prismatic, squamous, or pseudostratified, i.e. a layer of closely juxtaposed cells or contiguous cells, the majority of the cells of which contact the apical surface and the basolateral surface of the layer).

For the purposes of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

For the purposes of the invention, the term “mutant cell” means a cell carrying at least one mutation.

For the purposes of the invention, “progenitor cell” means a stem cell that is already engaged in cell differentiation but that has not yet differentiated.

For the purposes of the invention, “embryonic stem cell” means a pluripotent stem cell of cells derived from the internal cell mass of the blastocyst. The pluripotency of the embryonic stem cells can be evaluated by the presence of markers such as the transcription factors OCT4, NANOG and SOX2 and surface markers such as SSEA3/4, Tra-1-60 and Tra-1-81. The embryonic stem cells used in the context of the invention are obtained without destroying the embryo from which they originate, for example using the technique described in Chang et al. (Cell Stem Cell, 2008, 2(2)): 113-117). Optionally, embryonic stem cells from humans can be excluded.

For the purposes of the invention, “pluripotent stem cell” or “pluripotent cell” means a cell which has the capacity to form all the tissues present in the entire organism of origin, without however being able to form an entire organism per se. Human pluripotent stem cells can be called hPSC in the present application. These may in particular be induced pluripotent stem cells (iPSC or hiPSC for human induced pluripotent stem cells), embryonic stem cells or MUSE cells (for “multilineage-differentiating stress enduring”).

For the purposes of the invention, “induced pluripotent stem cell” means a pluripotent stem cell induced to become pluripotent by genetic reprogramming of differentiated somatic cells. These cells are in particular positive for pluripotency markers, such as staining with alkaline phosphatase and expression of the proteins NANOG, SOX2, OCT4 and SSEA3/4. Examples of methods for obtaining induced pluripotent stem cells are described in the articles by Yu et al. (Science 2007, 318 (5858): 1917-1920), Takahashi et al (Cell, 207, 131(5): 861-872) and Nakagawa et al (Nat Biotechnol, 2008, 26(1): 101-106).

For the purposes of the invention, “cell layer” means a monolayer of cells or an epithelial layer.

For the purposes of the invention, “cyst” means a three-dimensional spherical arrangement as a monolayer of cells or as an epithelial layer, surrounding a central lumen.

“Feret diameter” of a microcompartment (or of a part of a microcompartment) according to the invention means the distance “d” between two tangents to said microcompartment (or to said part), these two tangents being parallel, such that the entire projection of said microcompartment (or of said part) is between these two parallel tangents. A Feret diameter of the internal part of the microcompartment is measured between two interfaces of the internal part and the external layer of the microcompartment, i.e. the distance “d” between two tangents to said internal part, these two tangents being parallel, such that the entire projection of said internal part is between these two parallel tangents.

For the purposes of the invention, “variable thickness” of a layer means that, for a single microcompartment, the layer does not have the same thickness throughout.

For the purposes of the invention, “microcompartment” or “capsule” means a partially or entirely closed three-dimensional structure containing several cells.

For the purposes of the invention, “convective culture medium” means a culture medium stirred by internal movements.

For the purposes of the invention, “mutation” means a genetic or epigenetic mutation, preferentially a functional mutation. This may in particular be a point modification of the genetic sequence, a structural variant, an epigenetic modification, or a modification of the mitochondrial DNA.

For the purposes of the invention, “functional mutation” means a transmissible genetic or epigenetic modification which confers a gain or loss of function or a potential loss of function upon the mutant cell in question. This is preferentially a mutation causing a modification of the phenotype of the mutant cell in question. Very preferentially, it is a change in the genomic and/or epigenomic sequence which adversely affects the therapeutic potential of a population of cells, either by increasing the risk associated with the therapy produced, or by decreasing the benefit provided by the therapy produced.

For the purposes of the invention, “largest dimension” of a microcompartment or of a cell duster or of a layer of cells means the value of the largest Feret diameter of said microcompartment.

For the purposes of the invention, “smallest dimension” of a microcompartment or of a cell layer means the value of the smallest Feret diameter of said microcompartment.

For the purposes of the invention, “tissue” or “biological tissue” has the common meaning for tissue in biology, that is to say, the intermediate organization level between cell and organ. A tissue is an assembly of similar cells of the same origin (most commonly derived from a common cell line, although they can originate in the association of distinct cell lines), grouped into a cluster, network or bundle (fibre). A tissue forms a functional assembly, that is to say that its cells contribute to the same function. Biological tissues regenerate regularly and are assembled together to form organs.

For the purposes of the invention, “lumen” means a volume of aqueous solution topologically surrounded by cells. Preferentially, its content is not in diffusive equilibrium with the volume of convective liquid present outside the microcompartment.

“Smallest radius” of the internal part of a microcompartment according to the invention means half of the value of the smallest Feret diameter of the internal part of the microcompartment.

“Average radius” of the internal part of a microcompartment according to the invention means the average of the radii of the smallest compartment, each radius corresponding to half the value of a Feret diameter of the internal part of the microcompartment.

Cellular Microcompartments

The object of the invention is therefore a three-dimensional microcompartment 10, of ovoid, cylindrical, spheroid or spherical shape, or substantially ovoid, cylindrical, spheroid or spherical shape, comprising an external hydrogel layer 12 defining an internal part 14, said internal part 14 comprising at least:
extracellular matrix elements 16, and
at least two cysts, each cyst being formed by at least one layer of cells 18 organised three-dimensionally around a lumen 20.

Preferentially, the microcompartment is characterised in that the smallest radius of the internal part 14 is at least 100 μm, preferentially at least 200 μm, in particular between 200 and 400 μm.

The microcompartment according to the invention comprises an external hydrogel layer. Preferentially, the hydrogel used is biocompatible, that is to say it is non-toxic to the cells. The external hydrogel layer must allow the diffusion of oxygen and nutrients in order to supply the cells contained in the microcompartment and to enable them to survive. According to one embodiment, the external hydrogel layer comprises at least alginate. It may consist exclusively of alginate. The alginate can in particular be a sodium alginate, composed of 80% α-L-guluronate and 20% β-D-mannuronate, with an average molecular weight of 100 to 400 kDa and a total concentration of between 0.5 and 5% by weight. The external hydrogel layer has no cells.

The external hydrogel layer makes it possible in particular to protect the cells from the outside environment, to limit the uncontrolled proliferation of cells, and their differentiation in case of differentiation.

The average thickness of the external layer 12 may be variable. It is preferentially between 5 and 100 μm, more preferentially between 20 and 60 μm. The ratio between the smallest radius of the internal part 14 and this thickness is preferentially between 2 and 10.

The presence of an external hydrogel layer and extracellular matrix elements (preferentially an extracellular matrix) enables a uniform distribution of the cells between the microcompartments. Moreover, this external hydrogel layer makes it possible to prevent microcompartments from fusing, these fusion events being a major source of variability which is unfavorable for the phenotypic homogeneity of the cells.

The internal part 14, inside the external hydrogel layer, therefore comprises at least
extracellular matrix elements 16, preferentially in the form of an isotonic solution comprising extracellular matrix elements, in particular a biological or synthetic extracellular matrix, such as, for example, Matrigel®, and
at least two cysts, each cyst being formed by at least one layer of cells 18 organised three-dimensionally around a lumen 20.

The extracellular matrix elements preferentially comprise peptide or peptidomimetic sequences capable of binding to integrins. They are preferentially located in a layer between the cysts of cells and the external hydrogel layer 12. These extracellular matrix elements were preferentially added during the manufacture of the microcompartment and/or they were added to the microcompartment a posteriori and/or they were secreted or induced by the other constituents of the microcompartment.

The extracellular matrix elements preferentially comprise a mixture of extracellular proteins and compounds necessary for the culture and amplification of the cells. Preferentially, the extracellular matrix elements comprise structural proteins, such as collagen, laminins, entactin, vitronectin, and growth factors, such as TGF-beta and/or EGF. According to one variant, the internal part 14 may consist of or comprise Matrigel® and/or Geltrex® and/or a hydrogel-type matrix of plant origin, such as modified alginates, or of synthetic origin, or copolymer of poly(N-isopropylacrylamide) and poly(ethylene glycol) (PNIPAAm-PEG), of Mebiol® type. The presence of extracellular matrix elements promotes the adhesion of the initially encapsulated cells and contributes to obtaining and maintaining, in the microcompartment, at least two separate cell sources that do not meet, in order for each to form a cyst.

According to a preferred embodiment, the internal part 14 comprises an extracellular matrix.

If the microcompartment comprises extracellular matrix in the internal part 14, this may be extracellular matrix secreted by cells present in the microcompartment and/or extracellular matrix added at the time of the preparation/production of the microcompartment.

According to one variant, the solution containing extracellular matrix elements, in particular if it is a natural (biological) or synthetic extracellular matrix, may form a gel.

At the surface of the solution containing the extracellular matrix elements (preferentially a natural or synthetic extracellular matrix) in contact with a layer of cells, the solution containing the extracellular matrix elements (preferentially a natural or synthetic extracellular matrix) may optionally contain one or more cells.

Preferentially, the solution containing the extracellular matrix elements (preferentially a natural or synthetic extracellular matrix) has a Young's modulus of between 0.05 and 3 kDa. The Young's modulus can be measured by any method known to a person skilled in the art, in particular by measuring the rheology of gels of the same composition as the intermediate layer or else by AFM (atomic force microscopy).

A solution containing the extracellular matrix elements (preferentially a natural or synthetic extracellular matrix) with such Young's modulus values makes it possible to improve the maintenance of the cell phenotype and the genomic integrity of the cells contained in this intermediate layer during cell divisions.

In addition, such Young's modulus (elasticity) values promote the adhesion of the initially encapsulated cells and contribute to obtaining and maintaining, in the microcompartment, at least two separate cell sources that do not meet, in order for each to form a cyst. The viscosity of the solution containing the extracellular matrix elements (preferentially a synthetic or natural extracellular matrix) also promotes the adhesion of the initially encapsulated cells and contributes to obtaining and maintaining, in the microcompartment, at least two separate sources cells which do not meet, in order for each to form a cyst.

The internal part 14 may also comprise liquid areas without extracellular matrix elements. These liquid areas result from the equilibration by diffusion of the liquids present during the culture (initial seeding and change/renewal of any media). This liquid area is preferentially mainly composed of culture media. Advantageously, the external layer of the microcompartment keeps (at least partially) the factors and elements secreted by the cells near said cells, which reinforces the paracrine and autocrine effects within the microcompartment.

The internal part 14 of the microcompartment according to the invention comprises at least two cysts, each cyst being formed by at least one layer of cells 18 organised three-dimensionally around a lumen 20.

Each cyst is formed by at least one layer of cells organised three-dimensionally around a lumen, these human or animal cells preferentially excluding human embryonic stem cells. These cells are cells capable of forming a cyst, i.e. polarised cells with a basal surface capable of forming tight junctions and expressing podocalyxin on the apical surface (facing the lumen of the cyst). In a preferred embodiment, each cyst is formed by at least one layer of epithelial cells or cells having epithelial-type morphology. In a preferred embodiment, the cells of each layer 18 are epithelial cells or cells having an epithelial-type morphology and capable of forming a cyst.

Preferentially, each cyst is formed by at least one layer of human or animal cells, organised three-dimensionally around a lumen, chosen from induced pluripotent stem cells (iPSC) and the following cells: glandular epithelial cells (e.g. mammary or salivary), renal epithelial cells, intestinal epithelial cells (enterocytes), skin epithelial cells (keratinocytes), retinal pigment epithelial cells, epicardial cells, and endocardial cells.

A pluripotent stem cell, or pluripotent cell, refers to a cell that has the ability to form all the tissues present in the entire organism of origin, without however being able to form an entire organism per se. The pluripotent stem cells can in particular be induced pluripotent stem cells (IPS), MUSE (“Multilineage-differentiating Stress Enduring”) cells that are found in skin and bone marrow of adult mammals, or embryonic stem cells (ES).

Preferably, the cells constituting each cyst are polarised. The polarity of these cells inside each cyst can be demonstrated by the TJP-1 or ZO-1 proteins, both located on the internal/apical surface of the layer of pluripotent cells, which adjoins the lumen.

If the cells encapsulated in the microcompartment are intended to be used in cell therapy in human beings, the cells can be immuno-compatible with the person intended to receive them in order to avoid any risk of rejection.

The cells present in the microcompartment carry few, or even no, functional mutations.

The microcompartment according to the invention may contain at least 80, preferentially at least 800, at least 1000, at least 5000, in particular at least 8000 cells, these cells being organised in the form of at least two cysts.

Each cyst formed by at least one layer of cells within the microcompartment is hollow, i.e. it comprises an opening or lumen. The lumen is preferentially generated, at the time of the formation of the cyst, by the secretion of podocalyxin by the cells.

The lumen of each cyst can contain a liquid, in particular culture medium and/or a liquid secreted by the cells. Advantageously, the presence of this hollow part enables the cells to have a small diffusive volume of which they can control the composition, promoting cellular communication. Moreover, the internal part 14 is in equilibrium with the environment external to the microcompartment but may advantageously be enriched by the action of metabolic and/or secreted elements on the cells.

The cyst-shaped conformation makes it possible to reduce the pressures experienced by the cells compared to 2D cultures or aggregates. This configuration makes it possible to reduce cell mortality, and to increase the culture amplification factor. Consequently, this makes it possible to reduce the number of passes and dissociations required, to reduce the culture time necessary to reach the final required number of cells. Collectively, these improvements also contribute to maintaining the genetic integrity of the cells in the microcompartments.

Advantageously, the presence of several cysts in the same microcompartment makes it possible to improve the reproducibility of the encapsulation process and thereby the reproducibility of the cell batches, improves encapsulation survival and improves amplification per unit time.

The microcompartment may also comprise, in addition to cysts, cells in suspension in the microcompartment.

According to one embodiment, each cyst present in the microcompartment has been obtained by the encapsulation, in the internal part 14 of the microcompartments: of from 1 to 30 cells, preferentially from 1 to 10, in particular from 2 to 30 or from 3 to 30, in particular from 2 to 10 or from 3 to 10, more preferentially from 1 to 5, even more preferentially from 2 to 5 or from 3 to 5, preferentially at least 4 cells, and the smallest radius of the internal part 14 being at least 100 μm, preferentially at least 200 μm. The encapsulation of a controlled concentration of cells in a large capsule (smallest radius of the internal part of the microcompartment of at least 100 μm) contributes to obtaining at least two cysts in the microcompartment. It is difficult to obtain two cysts with a single cell, and it is preferable to encapsulate at least 2 cells, preferentially at least 3, even more preferentially at least 4, in the internal part 14 of the microcompartments. The encapsulation of an initial number of cells greater than 30 is possible but less advantageous, because it is difficult or even impossible (the probability drops) to obtain two cysts with more than 20/25% by volume of cells in the internal part at the time of encapsulation, in particular because this limits the amplification capacity to X4, which is disadvantageous for the production of cell batches. According to the invention, it is preferable that there be at least two separate cell sources that do not meet, in order for each to form a cyst.

The cells present in the microcompartment according to the invention were preferably obtained after at least two cell division cycles after encapsulation, in an external hydrogel layer, of at least one cell.

Preferably, the cells present in the microcompartment according to the invention were obtained after at least 2, 3, 4, 5, 6, 7, 8, 9, 10, 11, 12, 13, 14, 15, 20, 25, 28, 30 cell division cycles after encapsulation, in an external hydrogel layer, of preferentially at least 2 cells, preferentially at least 3, even more preferentially at least 4, in particular between 2 and 30 cells. For example, the cells present in the microcompartment were obtained after at least six cell division cycles after the encapsulation of the cells in the external hydrogel layer.

Preferentially, the number of cell divisions for the implementation of the method according to the invention is less than 300, even more preferentially less than 200.

Preferentially, the microcompartment is obtained after at least 2 passes after encapsulation, more preferentially at least 3, 4, 5, 6, 7, 8, 9 or 10 passes. Each pass can last for example between 2 and 15 days, in particular between 3 and 10 days.

Preferably, the microcompartment is obtained after at least one re-encapsulation, more preferentially between 1 and 14 re-encapsulations, in particular between 2 and 7 re-encapsulations. Very preferentially, a re-encapsulation corresponds to a new pass and each encapsulation cycle corresponds to a pass.

Preferentially, all of the cells initially encapsulated in the microcompartment before the first cell division cycle represents a volume less than 50% of the volume of the microcompartment in which they are encapsulated, more preferentially less than 40%, 30%, 20%, 10% of the volume of the microcompartment in which they are encapsulated.

Thus, according to one embodiment, the cells present in the microcompartment according to the invention were obtained after at least 2, 3, 4, 5, 6, 7, 8, 9, 10, 11, 12, 13, 14, 15, 20, 25, 28, 30 cell division cycles, after encapsulation in an external hydrogel layer of cell(s) representing a volume less than 50% of the volume of the microcompartment in which they are encapsulated, more preferentially less than 40%, 30%, 20%, 10% of the volume of the microcompartment in which they are encapsulated.

Preferentially, in the microcompartment according to the invention, the cells represent more than 50% by volume relative to the volume of the microcompartment, even more preferentially more than 60%, 70%, 75%, 80%, 85%, 90% by volume relative to the volume of the microcompartment.

In the microcompartment according to the invention, the volume of the internal part 14 preferentially represents at least 20% of the total volume of the microcompartment, preferentially at least 40%. This makes it possible to accommodate more cells after amplification, therefore more cysts and/or larger cysts.

The thickness of the layer of cells forming each cyst is preferentially between 6 and 200 μm, more preferentially between 6 and 60 μm.

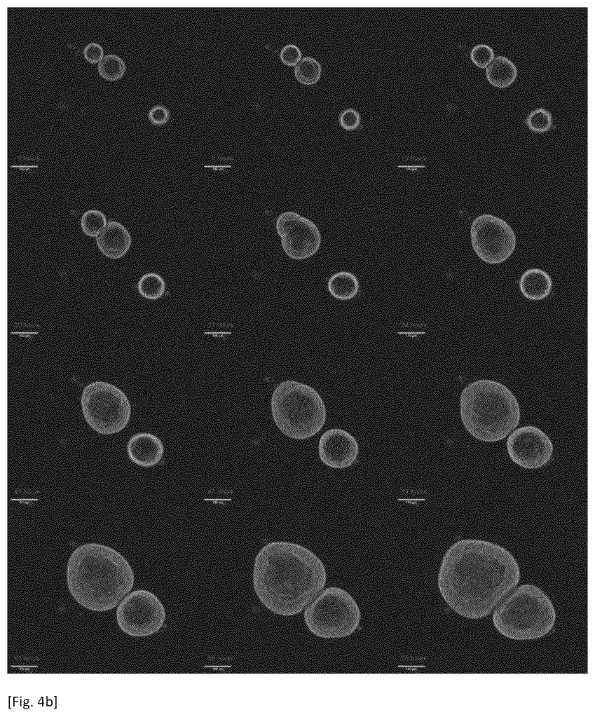

According to a variant shown in FIG. 4a and in FIG. 4b, at least two cysts can fuse together. In this case, the microcompartment according to the invention comprises at least one cyst coming from the fusion of two cysts. The cellular microcompartment according to the invention is closed or partially dosed, that is to say that the external layer is dosed or partially closed. Preferentially, the microcompartment is closed.

The microcompartment according to the invention can be in any three-dimensional form, that is, it may have the shape of any object in space. The microcompartment may have any shape compatible with cell encapsulation. Preferentially, the microcompartment according to the invention is in a spherical or elongated or substantially spherical or elongated shape. It may have the shape of an ovoid, a cylinder, a spheroid or a sphere or substantially this shape.

It is the external layer of the microcompartment, that is the hydrogel layer, which imparts the size and shape to the microcompartment according to the invention. Preferentially, the smallest dimension of the microcompartment according to the invention is between 202 μm and 1 mm, preferentially between 202 μm and 700 μm, even more preferentially 202 μm and 600 μm, in particular between 202 μm and 500 μm.

Its largest dimension is preferentially greater than 202 μm, more preferentially between 202 μm and 1 m, even more preferentially between 202 μm and 50 cm.

The invention also relates to a plurality of microcompartments together.

The invention also relates to an assembly or series of microcompartments comprising at least two three-dimensional cellular microcompartments, characterised in that at least one microcompartment is a microcompartment according to the invention.

Preferably, the series of microcompartments according to the invention is in a culture medium, in particular in an at least partially convective culture medium. Any culture medium suitable for culturing cells can be used, and in particular phosphate-buffered saline, such as for example “dulbecco's modified eagle medium” or “Rowell Park Memorial Institute medium”, as long as the concentration of dissolved salts is compatible with maintaining the crosslinking of the alginate by the divalent cations.

According to a particularly suitable embodiment, the object of the invention is a series of cellular microcompartments in a closed chamber, such as a bioreactor, preferentially in a culture medium in a closed chamber, such as a bioreactor. Thus, preferentially, the microcompartments are arranged in a culture medium in a closed bioreactor.

The assembly or series of microcompartments according to the invention preferentially comprises between 2 and 10¹⁶ microcompartments.

The microcompartments according to the invention may be used for all applications, in particular as a medication in cell therapy in humans or animals.

The microcompartment according to the invention may optionally be frozen to be stored. It should then preferentially be thawed before it is used.

Method for Obtaining Microcompartments According to the Invention

The invention also relates to a method for preparing microcompartments according to the invention.

The method for preparing a microcompartment or an assembly of microcompartments according to the invention may comprise the following steps:
(a) incubating human or animal cells in a culture medium containing at least one cytoprotective factor, in particular an inhibitor of apoptosis and/or Rho/A kinases,
(b) mixing the cells from step (a) with extracellular matrix elements, in particular a biological or synthetic extracellular matrix,
(c) encapsulating the suspension of cells in a hydrogel layer so as to form a microcompartment of ovoid, cylindrical, spheroid or spherical shape or substantially ovoid, cylindrical, spheroid or spherical shape, comprising an external hydrogel layer defining an internal part, the smallest radius or average radius of said internal part being at least 100 μm;
(d) culturing the resulting microcompartments in an isotonic rinsing buffer, preferentially for less than 30 minutes, then in a culture medium, preferentially in a culture medium containing at least one cytoprotective factor, in particular an inhibitor of apoptosis and/or Rho/A kinases;
(e) preferentially rinsing the microcompartments, so as to remove the cytoprotective factor (inhibitor of apoptosis and/or Rho/A kinases), preferentially within 48 hours after encapsulation, even more preferentially within 24 hours;
(f) culturing the microcompartments for at least two cell division cycles (amplification), preferentially between 1 and 20 days, even more preferentially between 2 and 10 days, in particular between 5 and 7 days, in a culture medium without a cytoprotective factor (inhibitor of apoptosis and/or Rho/A kinases), and
(g) optionally recovering the resulting cellular microcompartments.

The number of cells encapsulated in each microcompartment in step c) is preferentially 1 to 30 cells, in particular 2 to 30 or 3 to 30, preferentially 1 to 10, in particular 2 to 10 or 3 to 10, more preferentially 1 to 5, even more preferentially 2 to 5 or 3 to 5, preferentially at least 4 cells. The encapsulation of a controlled concentration of cells in a large capsule (smallest radius of the internal part of the microcompartment of at least 100 μm) contributes to obtaining at least two cysts in the microcompartment. It is difficult to obtain two cysts with a single cell, and it is preferable to encapsulate at least 2 cells, preferentially at least 3, even more preferentially at least 4, in the internal part 14 of the microcompartments. The encapsulation of an initial number of cells greater than 30 is possible but less advantageous, because it is difficult or even impossible (the probability drops) to obtain two cysts with more than 20/25% by volume of cells in the internal part at the time of encapsulation, in particular because this limits the amplification capacity to X4, which is disadvantageous for the production of cell batches. According to the invention, it is preferable that there be at least two separate cell sources that do not meet, in order for each to form a cyst.

Any culture medium suitable for culturing cells, in particular pluripotent stem cells, can be used, and in particular phosphate-buffered saline such as the “Rowell Park Memorial Institute medium” or Mtestrl or E8 Essential, for example.

The cytoprotective factor may for example be one or more inhibitor(s) of RHO/ROCK (“Rho-associated protein kinase”) pathways and/or an inhibitor of apoptosis known to a person skilled in the art, or any other cytoprotective factor known to a person skilled in the art. The cytoprotective factor must make it possible to promote survival of the cells and, in the case of the presence of an extracellular matrix, the adhesion of the cells to the extracellular matrix at the time of formation of the external hydrogel layer around said extracellular matrix.

In the method according to the invention, all of the cells initially encapsulated in step (c) preferentially represents a volume less than 50% of the volume of the microcompartment in which they are encapsulated, more preferentially less than 40%, 30%, 20%, 10% of the volume of the microcompartment in which they are encapsulated.

The method according to the invention may comprise a step prior to step a) or b) of dissociation of the cells by chemical, enzymatic or mechanical dissociation. This step is very preferentially necessary because cells of epithelial type, in particular stem cells, are adherent cells.

The encapsulated cells are suspended in the form of single cells and/or clusters or assembly (assemblies) of at least two cells (“cluster(s)”). Preferably, when there are at least 3 cells, the single cell(s) represent less than 50% by number of all the cells initially encapsulated in step (b). Indeed, it is preferable to encapsulate clusters of cells since this reduces distortions in chromosome segregation and incorrect chromosome segregations and consequently reduces the appearance of new mutageneses and contributes to maintaining the genomic integrity of the cells, since isolated cells risk dying through a lack of cell-cell interaction, and complete dissociation of cells leads to increased genetic anomalies.

Preferentially, each cell cluster initially encapsulated in step (c) has a greatest dimension less than 20% of the greatest dimension of a microcompartment in which it is encapsulated, even more preferentially less than 10%. Indeed, the cell clusters must not have a size that is too large compared to the size of the microcompartment, since a dimension of these initial cell clusters that is too large could lead, during cell divisions, to premature cell confluence in the capsule; this premature confluence of all or part of the capsules could lead to an increase in intracellular pressures and lead to cellular stress, affecting cellular growth, but also chromosome segregation.

The method according to the invention comprises a step b) of mixing the cells with an extracellular matrix between step a) and step c). According to one variant, this step b) can optionally be implemented before step (a) or else simultaneously with the encapsulation in step (c).

The encapsulation step c) is implemented according to techniques known to a person skilled in the art Indeed, any method for producing cellular microcompartments containing extracellular matrix and cells inside a hydrogel capsule may be used for the implementation of the preparation method according to the invention. In particular, it is possible to prepare microcompartments by adapting the method and the microfluidic device described in Alessandri et al., 2016 (“A 3D printed microfluidic device for production of functionalized hydrogel microcapsules for culture and differentiation of human Neuronal Stem Cells (hNSC)”, Lab on a Chip, 2016, vol. 16, no. 9, pp. 1593-1604), in accordance with the steps described below.

Preferentially, step c) is implemented in a device capable of generating hydrogel capsules using a microfluidic chip. For example, the device may comprise syringe pumps for several solutions injected concentrically by virtue of a microfluidic injector, which makes it possible to form a jet which breaks up into droplets which are then collected in a calcium bath. According to a particularly suitable embodiment, two or three solutions are loaded on two or three syringe pumps:
a hydrogel solution, for example alginate,
optionally an isotonic intermediate solution, preferentially an isotonic solution containing no divalent cations such as Ca²⁺ or Mg²⁺, to avoid excessively premature crosslinking of the hydrogel in the injector, such as for example a sorbitol solution,
the solution coming from step b) comprising cells, culture medium and the extracellular matrix.

The three solutions are co-injected (simultaneously injected) concentrically by virtue of a microfluidic injector or microfluidic chip which makes it possible to form a jet that breaks up into droplets, the external layer of which is the hydrogel solution and the core of which is the solution from step b) comprising cells; these droplets are collected in a calcium bath which crosslinks and/or gels the alginate solution to form the shell.

To improve the monodispersity of the cellular microcompartments, the hydrogel solution is preferentially charged with a direct current at between 1 and 10 kV. A ring to ground may optionally be arranged at a distance from the tip of between 1 mm and 20 cm, preferentially 3 mm to 10 cm, even more preferentially 1 cm to 5 cm, from the tip in the plane perpendicular to the axis of the jet exiting the microfluidic injector (coextrusion chip), to generate the electric field.

Preferentially, the solution is maintained at a temperature below 4° C. before being injected, to prevent the solution from gelling due to the presence of extracellular matrix elements.

According to the invention, it is necessary to generate capsules, the internal part of which has an average radius or smaller radius of at least 100 μm. To generate capsules with such dimensions with a coextrusion chip (microfluidic injector or microfluidic chip), the invention in particular proposes modifying the flow rate of the coextruded solutions and the final opening of the co-extrusion chip. “Flow rate” means the flow rate of each solution arriving at the injector. “Final opening of the coextrusion chip” means the internal opening of the outlet channel of the chip.
for the flow rate: the method preferentially goes, for each coextruded solution for the standard capsule sizes known from the prior art, from a value of between 20 and 40 ml/h to a value of between 45 and 150 ml/h, preferentially between 45 and 110 ml per hour for a variant of the invention,
for the final opening diameter of the coextrusion chip (microfluidic injector), which goes from a value preferentially between 50 and 120 μm for the standard capsules sizes known from the prior art, to a diameter value of between 150 and 300 μm, preferentially between 180 and 240 μm.

Thus, according to a particular embodiment, the encapsulation step (c) is carried out using a microfluidic injector, the final opening diameter of which is between 150 and 300 μm, preferentially between 180 and 240 μm, and with the flow rate of each of the 3 solutions of between 45 and 150 ml/h, preferentially between 45 and 110 ml/h. According to one embodiment, this encapsulation is carried out by co-injection of three solutions:
a hydrogel solution,
an intermediate isotonic solution, for example a sorbitol solution,
the solution coming from step b) comprising cells, culture medium and the extracellular matrix,
concentrically via a microfluidic injector which makes it possible to form a jet at the outlet of the injector consisting of the mixture of the three solutions, said jet breaking up into droplets, said droplets being collected in a calcium bath which stiffens the hydrogel solution to form the external layer of each microcompartment, the internal part of each droplet consisting of the solution coming from step (b) comprising cells, culture medium and the extracellular matrix.

Very preferentially, steps (d), (e) and (f) are carried out under continuous or sequential stirring. This stirring is important because it maintains the homogeneity of the culture environment and avoids the formation of any diffusion gradient. For example, it allows homogeneous control of cellular oxygenation level; thus avoiding phenomena of hypoxia-related necrosis or hyperoxia-related oxidative stress. By avoiding an increase in cell mortality and/or oxidative stress, stirring contributes to maintaining the genetic integrity of the cells.

The method according to the invention is preferentially carried out in a dosed chamber such as a closed bioreactor.

The number of cell division cycles in step (f) is at least 2, 3, 4, 5, 6, 7, 8, 9, 10, 11, 12, 13, 14, 15 cell division cycles.

Preferentially, the microcompartment is obtained after at least 2 passes (here a pass corresponds to a complete cycle of steps (a), (b), and (e), optionally (c) and (d)), more preferably at least 3, 4, 5, 6, 7, 8, 9 or 10 passes. Each pass may last for example between 2 and 15 days, in particular between 3 and 8 days. In a preferred variant, the method according to the invention comprises at least one re-encapsulation of the cells after step (f), i.e. at least two encapsulation cycles. Preferentially, each encapsulation cycle corresponds to a pass. In this variant of the method (at least one re-encapsulation of the cells after step (e)), the number of cell divisions of the entire method (for all passes) is at least 3, 4, 5, 6, 7, 8, 9, 10, 11, 12, 13, 14, 15, 20, 25, 30 cell division cycles.

In a method according to the invention, there may be several re-encapsulations, preferentially between 1 and 100, in particular between 1 and 10 re-encapsulation(s).

Each re-encapsulation can comprise:
a step which consists in dissociating the microcompartment or the series of microcompartments in order to obtain a suspension of cells or a suspension of cell clusters; the external hydrogel layer may be removed in particular by hydrolysis, dissolution, piercing and/or breaking by any biocompatible means, that is to say means which are not toxic for the cells. For example, the elimination may be accomplished using phosphate-buffered saline, a divalent ion chelator, an enzyme such as alginate lyase if the hydrogel comprises alginate, and/or laser microdissection, and
a step of re-encapsulating all or part of the cells or cell clusters in a hydrogel capsule. Re-encapsulation is a means suitable for increasing the cell amplification resulting from the pluripotent step, and reducing the risks of mutation.

Re-encapsulation consists in removing the external hydrogel layer, preferentially in resuspending, in a partially or totally dissociated manner, the cells which were in the form of cysts in the microcompartments, and in re-implementing the steps of the method.

According to one embodiment, the re-encapsulation comprises the following steps:
(i) removing the external hydrogel layer,
(ii) resuspending the cells that were contained in the microcompartment so as to obtain single cells and/or at least one assembly or cluster of cells in a culture medium containing at least one cytoprotective factor,
(iii) mixing the cells coming from step (a) with extracellular matrix elements,
(iv) encapsulating the solution of cells in a hydrogel layer so as to form a microcompartment of ovoid, cylindrical, spheroid or spherical shape, or substantially ovoid, cylindrical, spheroid or spherical shape, comprising an external hydrogel layer defining an internal part, the smallest radius or average radius of said internal part being at least 100 μm;
(v) culturing the resulting microcompartments in a culture medium containing at least one cytoprotective factor, in particular an inhibitor of apoptosis and/or Rho/A kinases,
(vi) preferentially rinsing the microcompartments, so as to remove the cytoprotective factor;
(vii) culturing the microcompartments in a culture medium without a cytoprotective factor, for at least one cell division cycle, and
(viii) optionally recovering the resulting cellular microcompartments.

The compartmentalisation into microcompartments makes it possible to remove the microcompartments containing more mutated cells than the other capsules. Even if the mutated cells have rapid growth, they will reach capsular confluence which will limit their multiplication. The compartmentalisation also makes it possible not to contaminate the entire cell population, and also to remove the capsules containing mutant cells, at any time, in particular before a re-encapsulation step. This sorting may be done either by inline analysis, or by removing capsules which filled up more quickly than others, for example.

According to one variant of the invention, the encapsulated cells are differentiated cells that are reprogrammed into pluripotent cells inside the hydrogel capsule during the formation of the microcompartments. According to one variant, the cell reprogramming agents can be added in step (a) and/or (b) and/or (c) and/or (d) and/or (ii) and/or (iii) and/or (iv) and/or (v). Preferentially, they are cell reprogramming agents that do not permeate the hydrogel layer. The addition of reprogramming agents is particularly relevant when the initially encapsulated cells are differentiated cells that are to be dedifferentiated, in particular back to the pluripotent stage. A person skilled in the art knows how to reprogram a differentiated cell into a stem cell by reactivating the expression of genes associated with the embryonic stage by means of specific factors, designated in the present invention as “reprogramming agents”. The methods described in the following may be cited as examples: Takahashi et al., 2006 (“Induction of pluripotent stem cells from mouse embryonic and adult fibroblast cultures by defined factors” Cell, 2006 Vol 126, pages 663-676), Ban et al., 2009 (“Efficient induction of transgene-free human pluripotent stem cells using a vector based on Sendai virus, an RNA virus that does not integrate into the host genome” Proc Jpn Acad Ser B Phys Biol Sci. 2009; 85(8):348-62) and the international application WO2010/105311, the title of which is “Production of reprogrammed pluripotent cells”. The reprogramming agents are advantageously co-encapsulated with the differentiated cells, so as to concentrate the product and promote contact with all of the cells. In the case of reprogramming agents that permeate the hydrogel layer, it is possible to add said agents to the culture medium after the encapsulation step. Reprogramming agents make it possible to impose a succession of phenotypic changes on the cells, back to the pluripotent stage. Advantageously, the step of reprogramming is carried out using specific culture media, promoting these phenotypic changes. For example, the cells are cultured in a first medium comprising 10% of human or bovine serum, in an Eagle's minimum essential medium (DMEM) supplemented with a serine/threonine protein kinase receptor inhibitor (such as the product SB-431542 (C₂₂H₁₈N₄O₃)), one or more RHO/ROCK (RHO-associated protein kinase) pathway inhibitors, such as thaizovivin and/or Y-27632, fibroblast growth factors, such as FGF-2, ascorbic acid and antibiotics, such as Trichostatin A (C₁₇H₂₂N₂O₃). Then, the culture medium is replaced with medium promoting the multiplication of the pluripotent cells, such as mTeSR® 1 medium.

The incubation in steps (a) and/or (ii) is carried out preferentially for a time of between a few minutes and a few hours, preferentially between 2 minutes and 2 hours, more preferentially between 10 minutes and 1 hour.

Step (d) and/or (v) of culturing with a cytoprotective factor is carried out for a time of between 2 and 48 hours, preferentially for a time of between 6 and 24 hours, more preferentially for a time of between 12 and 18 hours.

The rinsing step can be carried out by one or more rinsing operations, in successive culture media without RHO/ROCK pathway inhibitors, in less than 48 hours, preferentially less than 24 hours, more preferentially between 12 and 18 hours after the start of step (d) and/or (v).

In one embodiment, at least one of the steps (preferentially all of the steps) is carried out at a temperature suited to the survival of the cells, of between 4 and 42° C. The temperature during cell proliferation must preferentially be between 32 and 37° C. to avoid triggering mutations by lowering the performance of repair enzymes. Likewise, preferably, the temperature must be low (ideally about 4° C.) to manage the stress on the cells in step (c).

At any time, the method according to the invention may comprise a step consisting of verifying the phenotype of the cells contained in the microcompartment. This verification can be carried out by identifying the expression, by at least some of the cells contained in the microcompartment, of at least one gene specific to the phenotype sought.

The cellular microcompartments obtained according to the methods of the invention can then be frozen before any use. The freezing is preferentially carried out at a temperature of between −190° C. and −80° C. The thawing can be carried out in a warm water bath (37 degrees preferentially) so that the cells thaw quite rapidly. The microcompartments according to the invention, before they are used, may be kept at more than 4° C. for a limited time before they are usd, preferentially between 4° C. and 38° C.

The implementation of the method according to the invention makes it possible to obtain microcompartments comprising at least 80, preferentially at least 800, at least 1000, at least 5000, in particular at least 8000 cells, these cells being organised in the form of at least two cysts.

Advantageously, the 3-dimensional structure of the cells in the microcompartment and the low or even zero percentage of cells isolated during encapsulation (most of the cells being encapsulated in the form of cell clusters), reduces chromosome desegregation and consequently reduces the appearance of new mutageneses.

The invention especially promotes amplification with a high amplification factor, which consequently reduces the culture time and the number of divisions to obtain a very large number of cells, and therefore limits new mutageneses.

The protection of the cells by virtue of the external layer and the presence of extracellular matrix elements reduces chromosome desegregation and limits the mechanical stress on the cells, and consequently reduces the appearance of new mutageneses.

Advantageously, the presence of several cysts in each microcompartment also makes it possible to promote initial cell survival and smooths out asynchronous growth.

The invention will now be illustrated using an example and results

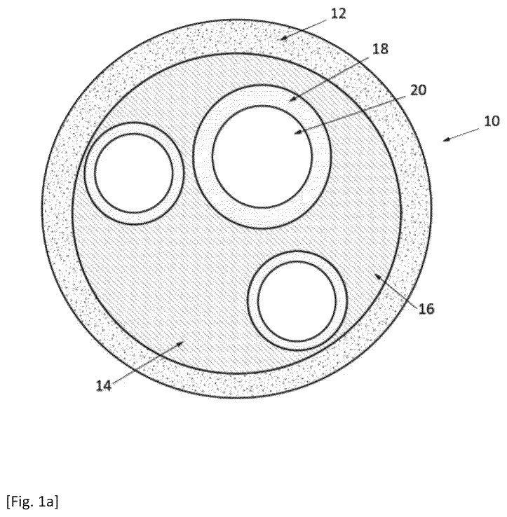

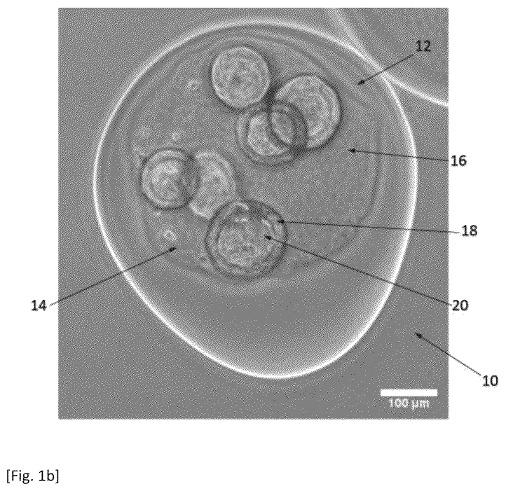

FIGS. 1a and 1b show hollow hydrogel capsules enabling the growth of several cysts of human pluripotent stem cells in the central microcompartment.

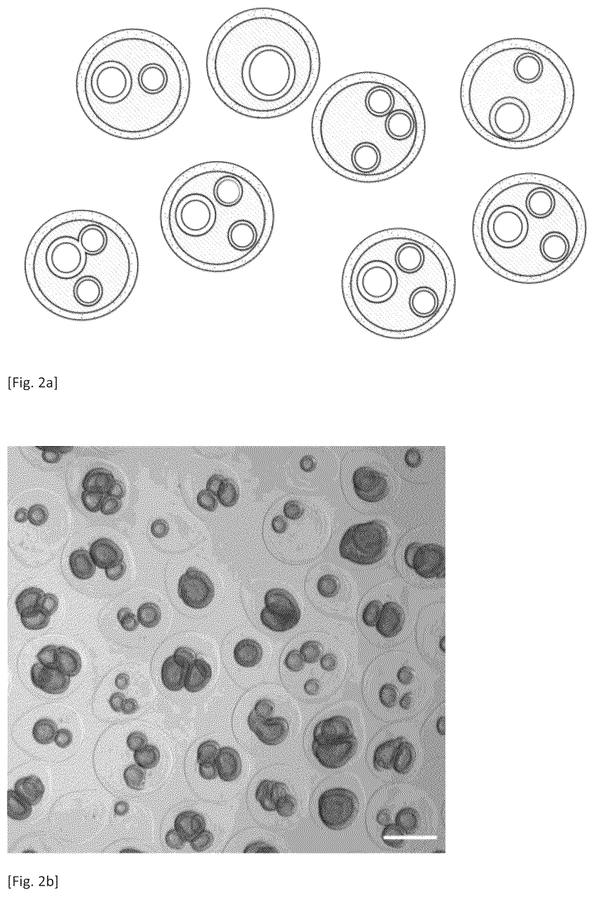

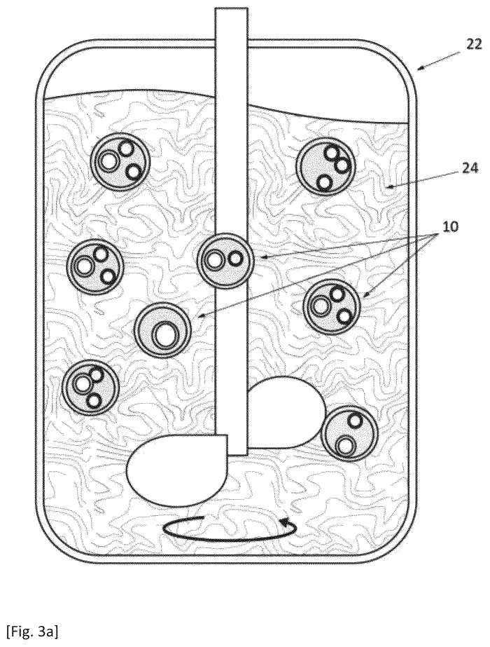

FIGS. 2a and 2b show a microscopy image, at day 6.5 post-encapsulation, showing a plurality of hollow alginate capsules containing human pluripotent stem cells. The scale bar is 500 μm. The microcompartments according to the invention comprise several 3D colonies of stem cells. These colonies all have a homogeneous cystic configuration with an average diameter of 170 μm. On average, 2.6 colonies (cysts) are observed per capsule at this stage of the culture.

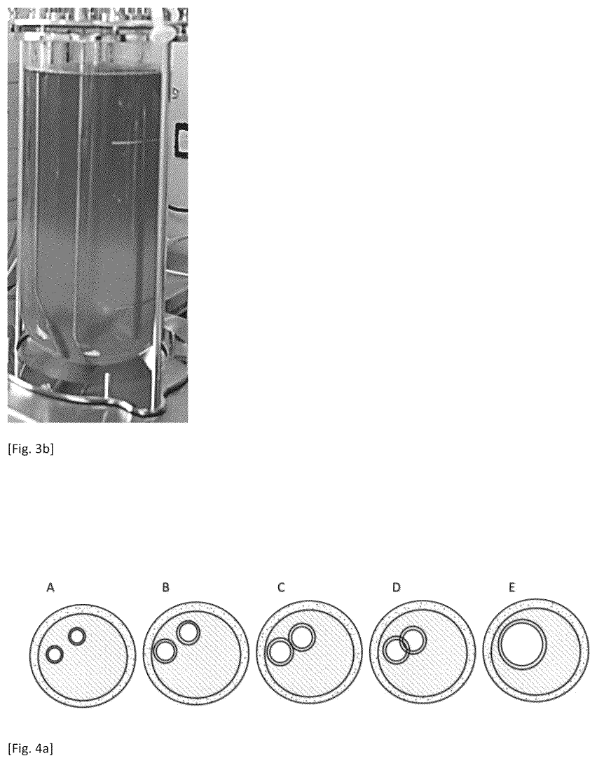

FIGS. 3 a and 3 b show a plurality of multi-cyst capsules cultured in suspension in a bioreactor.

The key parameters and results of an example of encapsulation according to the invention in a 10-litre bioreactor are given in table 1 below.

TABLE 1

| | 10 \| Bioreactor - Test 1 | 10 \| Bioreactor - Test 2 |
|---|---|---|
| Target oxygen threshold | 20% DO | 20% DO |
| Maximum volume | 10.21 | 9.71 |
| Min % capsule volume | 4.5-15% | 3.9 to 14% |
| Max cells/ml capsule | 33.92 m/ml | 33.80 m/ml |
| hPSCs seeded | 56 million | 51 million |
| hPSCs produced | 15.2 billion | 14.52 billons |
| Amplification factor (AF) | X271/6.59 days | X282/6.69 days |
| Doubling time (PDT) | 19.4 hours | 19.73 hours |
| Number of cycles (PDL) | 8.14 | 8.08 |
| Final viability | 99.5% | 99.7% |
| OCT4+ final | 92.0% | 96.7% |
| SOX2+ final | 99.6% | 99.7% |
| NANOG+ final | 97.0% | 98.4% |

The encapsulation of the hiPSCs and the culture in suspension were carried out for 2 separate cycles. The target oxygen level of 20% dissolved oxygen is described as a hypoxic condition (unlike bioreactors with a dissolved oxygen level of 100%, which are described as normoxic). A “fed batch” strategy, that is, a renewal of the culture medium by subtraction/addition of medium at regular intervals, was applied, causing an increase in the working volume and a decrease in the concentration of the capsules relative to the total volume. The cell density can be expressed in millions of cells per millilitre of capsules. The volume of the harvested capsule is measured in a graduated glass cylinder. The measured number of cells can be related to this previously measured capsule volume, in order to deduce therefrom a cell concentration per capsule volume. The amplification factor (AF) is defined such that AF=N(t0+Dt)/N(t0), where N(t0) and N(t0+Dt) are the number of initial cells at t0 and t0+Dt, respectively. The doubling level of the population (PDL) is defined as PDL(t)=ln(AF)/ln(2). The doubling time of the population (PDT) is defined such that PDT(t)=Dt/PDL where Dt is the culture time.

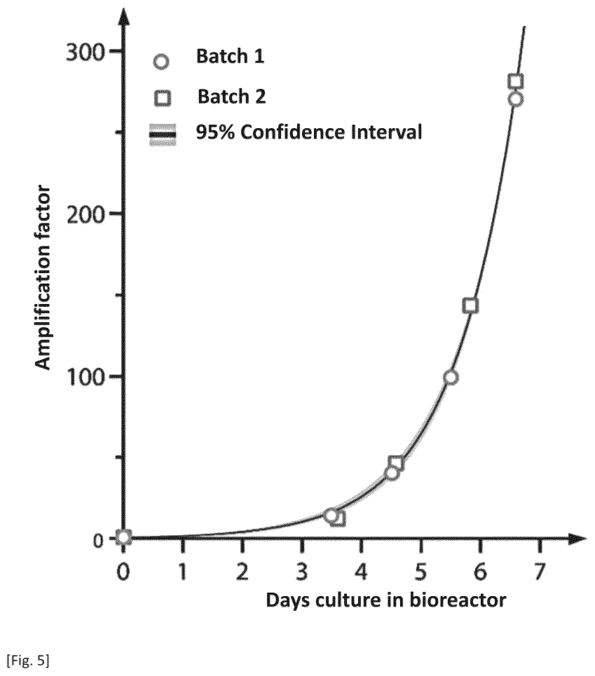

The viability was evaluated by the Nucleocounter NC-3000 (Chemometec). The decapsulated and dissociated cells were fixed and stained for OCT4, SOX2 and NANOG and analysed by BD Accuri C6 Plus flow cytometry. The results concerning the amplification factor are also presented in FIG. 5.

FIG. 4b shows a series of photos from a video microscopy of a capsule comprising several cysts according to the invention.

The observation is carried out in transmission with a Nikon bio-station IM with a 10× objective. The average external diameter of the quasi-spherical capsule is 288 μm as indicated by the 100 μm scale bar. The initial picture was taken 4 days after encapsulation. The sequence illustrates the growth of several colonies of pluripotent stem cells in a hollow hydrogel capsule. These colonies are cultured in a closed 3D microcompartment, externally defined by a crosslinked alginate layer. The internal compartment is described as being hollow, in the sense that it does not contain crosslinked alginate. The internal compartment containing the cells also contains extracellular matrix (here, Matgel®) which is visible in microscopy by a different granularity to that of alginate.

During the encapsulation, 50% of the volume injected into the internal microcompartments comes from the cell/matrix mix and 50% from an isomolar sorbitol solution (cf. method). Thus, the internal space is at least 50% liquid.

Initially, the 3 colonies have a homogeneous cystic configuration with respective diameters of 76 μm, 100 μm and 78 μm (the bottom-right cyst in the image). At this initial stage, the epithelial thickness of these cysts is respectively 10 μm, 13 μm and 12 μm. It is observed that the 2 cysts at the top left of the capsule gradually enter into contact and fuse their internal lumens as well as their epithelia. The result thereof is a colony of stem cells of greater size which also has a circular and symmetrical cystic structure.

54 hours after the start of the observation (right-hand images, penultimate line of FIG. 4b), the 2 cystic colonies present in the capsule come into contact. At this stage, the diameters of these cysts are 232 and 175 μm. The respective thicknesses of the cell layer of these cysts are 36 and 24 μm, respectively. It is observed that contact between these colonies is not followed by fusion of the lumens or epithelia. The limit between these 2 structures remains clearly identifiable on the last and 12th image (75th hour).

A shift of the largest of the colonies by 20 μm to the left is observed: this colony appears to be pushed away by the small colony. Despite a slight flattening of the contact zone between the 2 colonies, their symmetrical cystic structures do however remain for each of the 2 colonies, with homogeneous cell layer thicknesses of 50 and 40 μm, respectively. This growth of the encapsulated colonies and their apparent ability to push one another away in the inner capsular space is facilitated by the at least partially liquid nature of this internal hollow space. In addition, the use of gradually degradable extracellular matrices such as Matrigel also allows the cells to grow in a more permissive 3D environment than during encapsulation in a solid hydrogel bead, thereby making it possible to maintain a cystic epithelial structure despite the forces generated by cell multiplication.

In all, the partially liquid hollow intra-capsular space of this large capsule allows the 3D colonies of stem cells to grow while preserving a stable cystic epithelial phenotype. The stability of this phenotype allows good phenotypic homogeneity of the cells produced and a robust/reliable bioproduction method, illustrating the persistence of the epithelial phenotype during the growth of several cysts

FIG. 4a shows a “multi-cyst” fusion sequence in capsules:
A: Self-organisation and lumen formation of 3D colonies of stem cells inside the microcompartment. Formation of the encapsulated stem cell cysts
B: Growth of the stem cell cysts: gradual increase in the diameter of the cyst, diameter of the lumen and thickness of the cyst.
C: The cysts come into contact
D: The cysts fuse their cell layers
E: The cysts also fuse their internal lumens, resulting in a single cyst of larger size.

1. A three-dimensional microcompartment (10) of ovoid, cylindrical, spheroid or spherical shape or substantially ovoid, cylindrical, spheroid or spherical shape, comprising an external hydrogel layer (12) defining an internal part (14), said internal part (14) comprising at least:
extracellular matrix elements (16), and
at least two cysts, each cyst being formed by at least one layer of human or animal cells (18), excluding human embryonic stem cells, organised three-dimensionally around a lumen (20),
the smallest radius of the internal part (14) being at least 100 μm.

2. The three-dimensional microcompartment (10) according to claim 2, characterised in that the cells of each layer (18) are epithelial cells or cells having epithelial-type morphology and capable of forming a cyst.

3. The three-dimensional microcompartment (10) according to claim 1, characterised in that the cells of each layer (18) are chosen from induced pluripotent stem (iPSC) cells and the following cells: glandular epithelial cells, renal epithelial cells, intestinal epithelial cells, skin epithelial cells, retinal pigment epithelial cells, epicardial cells, and endocardial cells.

4. The three-dimensional microcompartment (10) according to claim 1, characterised in that the internal part (14) also comprises liquid areas without extracellular matrix elements.

5. The three-dimensional microcompartment according to claim 1, characterised in that the smallest radius of the internal part (14) is at least 200 μm.

6. The three-dimensional microcompartment according to claim 1, characterised in that the volume of the internal part (14) represents at least 20% of the total volume of the microcompartment, preferentially at least 40%.

7. The three-dimensional microcompartment according to one of the preceding claims, characterised in that it is closed.

8. The three-dimensional microcompartment according to claim 1, characterised in that the external layer comprises alginate.

9. The three-dimensional microcompartment according to one of the preceding claims, characterised in that at least one cyst comes from the fusion of two cysts.

10. The three-dimensional microcompartment according to claim 1, characterised in that the cells present in the microcompartment were obtained by the encapsulation, in the internal part of an external hydrogel layer, of 2 to 30 cells.

11. The three-dimensional microcompartment according to claim 1 for use thereof as a medication.

12. An assembly of microcompartments comprising at least two three-dimensional cellular microcompartments, characterised in that at least one microcompartment is a microcompartment according to claim 1.

13. The assembly of microcompartments according to claim 12, characterised in that the microcompartments are arranged in a culture medium in a bioreactor.

14. A method for preparing a cellular microcompartment according to claim 1 or an assembly comprising the microcompartment, the method comprising the following steps:
(a) incubating human or animal cells in a culture medium containing at least one cytoprotective factor,
(b) mixing the cells from step (a) with extracellular matrix elements, preferentially a biological or synthetic extracellular matrix,
(c) encapsulating the suspension of cells in a hydrogel layer so as to form a microcompartment of ovoid, cylindrical, spheroid or spherical shape or substantially ovoid, cylindrical, spheroid or spherical shape, comprising an external hydrogel layer defining an internal part, the smallest radius or average radius of said internal part being at least 100 μm;
(d) culturing the resulting microcompartments in an isotonic rinsing buffer, then in a culture medium, preferentially in a culture medium containing at least one cytoprotective factor,
(e) preferentially rinsing the microcompartments, so as to remove the cytoprotective factor;
(f) culturing the microcompartments for at least two cell division cycles (amplification), preferentially between 1 and 20 days, even more preferentially between 2 and 10 days, in particular between 5 and 7 days, in a culture medium without a cytoprotective factor, and
(g) optionally recovering the resulting cellular microcompartments.

15. The method according to claim 14, characterised in that step c) is carried out by co-injection of two or three solutions:
a hydrogel solution,
optionally an isotonic intermediate solution,
the solution coming from step b) comprising cells, culture medium and the extracellular matrix,
concentrically via a microfluidic injector which makes it possible to form a jet at the outlet of the injector consisting of the mixture of the solutions, said jet breaking up into droplets, said droplets being collected in a calcium bath which stiffens the hydrogel solution to form the external layer of each microcompartment, the internal part of each droplet consisting of the solution coming from step (b) comprising cells, culture medium and the extracellular matrix.

16. The method according to claim 15, characterised in that the final opening diameter of the microfluidic injector is between 150 and 300 μm, preferentially between 180 and 240 μm, and the flow rate of each of the solutions is between 45 and 150 mi/h, preferentially between 45 and 110 ml/h.

17. The method according to claim 14, characterised in that all of the cells initially encapsulated in step (c) represents a volume less than 50% of the volume of the microcompartment in which they are encapsulated.

18. The method according to claim 14, characterised in that step b) of mixing the cells with an extracellular matrix is carried out either between step (a) and step (c), or simultaneously with the encapsulation in step (c).

19. The method according to claim 14, characterised in that steps (d), (e) and (f) are carried out under continuous or sequential stirring.

20. The method according to claim 14, characterised in that it is implemented in a bioreactor.

21. The method according to claim 14, characterised in that, prior to or simultaneously with step (a), the method comprises a step of dissociation of the cells by chemical, enzymatic or mechanical dissociation.

22. The method according to claim 14, characterised in that the method comprises at least one re-encapsulation of the cells after step (f).

23. The method according to claim 22, characterised in that each re-encapsulation corresponds to a pass.

24. The method according to claim 22, characterised in that each re-encapsulation consists in removing the external hydrogel layer, preferentially in resuspending, in a partially or totally dissociated manner, the cells which were in the form of cysts in the microcompartments, and in re-implementing the steps of the method.
